(19) United States
(12) Patent Application Publication
(10) Pub. No.: US 2022/0387698 A1
(43) Pub. Date: Dec. 8, 2022

(54) SELF-PRESSURIZING FLUID DELIVERY SYSTEMS AND DEVICES AND METHODS OF USING THE SAME

(71) Applicant: Nathan GLASS, Fortville, IN (US)

(72) Inventor: Nathan Glass, Fortville, IN (US)

(21) Appl. No.: 17/770,032

(22) PCT Filed: Oct. 19, 2020
(86) PCT No.: PCT/US20/56380

§ 371 Date: Apr. 18, 2022

Related U.S. Application Data
(60) Provisional application No. 62/923,114, filed on Oct. 18, 2019.

(51) Int. Cl. A61M 3/02; B65D 81/05

(52) U.S. Cl. CPC A61M 3/0237; B65D 81/052

(57) ABSTRACT

Self-pressurizing fluid deliver systems and devices and methods of using the same. An exemplary pressurized sterile fluid storage bag system referenced herein includes a foam cavity formed from a rigid, non-conforming material and an elastic, conforming material and containing a resilient open cell or closed cell foam with capabilities to restore form when compressed, an air valve, a fluid cavity formed from said elastic, conforming material on one side and a material which may be rigid and non-conforming or elastic and conforming, and a fluid valve.

### PRIORITY

The present application is related to, and claims the priority benefit of, U.S. Provisional Patent Application Ser. No. 62/923,114, filed Oct. 18, 2019, the contents of which are incorporated herein directly and by reference in their entirety.

### BACKGROUND

### Field of the Invention

The present disclosure relates generally to devices, such as packages and bags, for dispensing sterilized fluids in a medical setting. Specifically, the present disclosure relates to devices and systems configured to and capable of pressurizing interior components to force fluid contents to an ejection port.

### Background Art

In many surgical, medical diagnostic, and therapeutic procedures, physicians or medical personnel inject patients with a pressurized, sterile fluid. Some examples of these procedures include wound irrigation, cancer treatment through chemotherapy drugs, rehydration through saline administration, and fluoroscopy procedures.

The simplest form of pressurizing fluid is by using the force of gravity. Such is the method in hanging a sterile fluid bag from an intravenous (IV) pole, which is the most common pressurized fluid delivery system. This system typically involves three components: a sterile fluid bag, a length of tubing, and a fluid delivery port. The sterile fluid bag is connected directly to the length of tubing, which is then connected to the fluid delivery port. The sterile fluid bag is hung at an elevation higher than its fluid delivery port such that the force of gravity establishes a pressurized system. Though this method is simple to achieve, it has several disadvantages. The primary concern for this design is the lack of a constant pressure gradient. As fluid flows to the delivery site, the mass of fluid in the bag decreases, thereby decreasing the force of gravity which pressurizes the system. This results in a gradual decrease in pressure and a corresponding decrease in the amount of fluid delivered, such that in many cases the sterile fluid bag may not fully drain to its desired site. Because of this concern, the bag must repeatedly be raised to higher elevations during a single use, which is quite inconvenient.

Due to the increasing specificity and accuracy required of minimally invasive percutaneous procedures, there exists a need to develop a more sophisticated system to deliver sterile, pressurized fluid to medical instruments or patients. Recent innovations related to pressurizing sterile fluid in a single system have been made, with most of these innovations involving an exterior source for achieving the force required to pressurize the fluid. Such is the case in U.S. Pat. No. 5,810,202, for example. That invention involves an interior, fluid containing bag and an exterior, air filled bag. A hand actuated pressure source inflates the exterior bag which establishes a uniform compressive force on the interior bag. An ejection port then allows the pressurized fluid to be released. Though this has the potential to be more consistent in its pressure gradient, the pressure must still be constantly maintained as the interior bag size will decrease as fluid is released.

Similarly, U.S. Pat. No. 5,163,909, U.S. Pat. No. 8,992,489, and U.S. Pat. No. 3,949,753 pressurize fluid via a dual cavity system and an external power source. However, these innovations involve pressurized gas which can be expensive and potentially dangerous. U.S. Patent Application Publication No. 2003/0135159 discloses devices that pressurize fluid through its own air intake system, but this device's inclusion of many small parts and an electrical system is expensive in comparison to existing technologies.

The present disclosure includes disclosure of devices and systems to apply the restoring expansion force of resilient foam to pressurize the system. This concept has been explored in existing innovations, such as U.S. Pat. No. 3,871,377, which discloses a device that utilizes the expansive force of foam as a suction force to evacuate waste fluids from surgical sights. Perhaps the most similar concept to the present invention is U.S. Pat. No. 5,176,641, which discloses the utilization of the restorative force of foam to pressurize liquid within the same cavity. However, this device is implant exclusive and implies direct contact of sterile liquid to a foam, which can result in undesirable or unforeseen fluid-foam interactions. Additionally, the force required to compress the foam is achieved through fluid injection, which can prove difficult for the end-user and has the potential of retrograde flow and splash back of fluid during loading.

### BRIEF SUMMARY

Systems of the present disclosure can comprise several elements. One such elements is a fluid cavity of a bag which can be created by sealing the perimeter of an elastic, fluid impermeable material (which henceforth be referred to as the “dividing barrier”) to the perimeter of another fluid impermeable material which may be rigid and nonconforming or elastic and conforming (which will henceforth be referred to as the “fluid barrier”). The sizing of this cavity may vary with respect to the amount of fluid necessary for the medical procedure and is therefore unspecified herein.

The fluid cavity is airtight aside from an additional element of systems of the present disclosure, namely the fluid valve port. This port passes through fluid barrier to allow flow of fluid both into and out of the fluid cavity. The fluid valve port is to feature a mechanism which allows for air locking. In its preferred embodiment, the fluid valve port mechanism will be adjustable such that the pressure can be changed to fit user preference. This can be achieved through varying the diameter of the fluid valve port. The fluid valve port is also designed such that it can be connected to tubing as well.

On the other side of the dividing barrier is an additional element of systems of the present disclosure, namely the foam cavity. This cavity is formed by sealing the perimeter of a rigid, nonconforming material (which will henceforth be referred to as the “foam insert”) to the perimeter of the dividing barrier. The foam cavity is to be filled with resilient, close cell or open cell foam. It is essential that the foam has characteristics such that it may return to its original form (or close to its original form) after being compressed. The specifications of the foam may vary depending on the amount of fluid pressure necessary and is therefore unspecified at this time.

The foam cavity is airtight aside from an additional component of systems of the present disclosure, namely the air valve port. This port passes through foam insert to allow flow of air both into and out of the foam cavity. The air valve port is to feature a mechanism which allows for air locking. In its preferred embodiment, the air valve port mechanism will be adjustable such that the allowance of air passage can be changed to fit user preference. This can be achieved through varying the diameter of the air valve port. The air valve port is designed such that it may connect to tubing to assist with air removal from the foam cavity.

Devices and systems of the present disclosure can be used as follows. The air valve port is to be opened to allow air to exit from the foam cavity. The foam is then compressed completely and such that there is minimal airspace in the foam cavity. The air valve port is then closed such that no air may flow into the foam cavity. The fluid valve port can be opened to allow sterile, medical fluid to be loaded into the fluid cavity. The system is designed such that there is minimal likelihood of retrograde flow or splash back during fluid loading. The compression of the foam followed by the locking of the air valve not only pressurizes the system, but also creates space for the fluid cavity to be filled. Once the foam cavity is compressed, sterile medical fluid can be loaded into its cavity with essentially no resistive force.

The fluid valve port can be closed, and the pressurized system is now ready for use. Prior to its use, the user may elect to connect various instruments or tubing to the fluid valve port. Opening of the air valve port results in air inflow to the foam cavity. This results in foam expansion, which is the driving force of the pressurized outflow of liquid. The rigidity of the foam insert disallows stretching, and as a result the force of the expanding foam pushes against the dividing barrier. Due to its elasticity, the dividing barrier stretches in the direction of the fluid cavity. The compressive force on the fluid cavity decreases its internal volume, creating pressure. Subsequent opening of the fluid valve port results in release of pressurized fluid contents. The systems of the present disclosure are designed such that the size of the fluid cavity continues to decrease even as fluid contents are released from the system, due to the continuous expansion of the foam. This results in a constant pressure gradient which does not have to be adjusted during use.

The present disclosure includes disclosure of a pressurized sterile fluid storage bag system, the system comprising a foam cavity formed from a rigid, non-conforming material and an elastic, conforming material and containing a resilient open cell or closed cell foam with capabilities to restore form when compressed, an air valve, a fluid cavity formed from said elastic, conforming material on one side and a material which may be rigid and non-conforming or elastic and conforming, and a fluid valve.

In at least one exemplary embodiment, the system further comprises a common seam to seal the perimeter of all cavity forming materials, such that said fluid cavity is adjacent to said foam cavity with the elastic, conforming material separating the two. In at least one exemplary embodiment, the said foam cavity is shaped such that the foam insert is not compressed when said air valve is open. In at least one exemplary embodiment, said foam cavity can be compressed in a matter which compresses its foam contents. In at least one exemplary embodiment, said rigid, nonconforming material forming said foam cavity does not stretch during expansion of foam insert contents.

In at least one exemplary embodiment, said air valve passes through the rigid, nonconforming material used to create said foam cavity and allows air passage between the ambient and the internal air space of said foam cavity. In at least one exemplary embodiment, said air valve features a locking mechanism which allows for complete air locking to fully disallow or permit passage of air between ambient and internal air space of said foam cavity. In at least one exemplary embodiment, said air valve features a locking mechanism which allows the user to adjust the diameter of air exit hole.

In at least one exemplary embodiment, said elastic, nonconforming material acts as the dividing barrier between said foam cavity and said fluid cavity. In at least one exemplary embodiment, said elastic, nonconforming material is able to stretch during expansion of said foam insert. In at least one exemplary embodiment, said fluid cavity is air tight and compresses in internal volume during expansion of said foam insert. In at least one exemplary embodiment, said fluid valve passes through the rigid, nonconforming material or elastic, conforming material used to form the outer surface of said fluid cavity and allows for fluid passage between the ambient and internal space of said fluid cavity.

In at least one exemplary embodiment, said fluid valve features a luer lock on its exterior portion to allow for connection to tubing or various instruments. In at least one exemplary embodiment, said fluid valve features a locking mechanism which allows for complete air locking to fully disallow or permit passage of fluid between the ambient and internal space of said fluid cavity. In at least one exemplary embodiment, said fluid valve features a locking mechanism which allows the user to adjust the diameter of the fluid exit hole. In at least one exemplary embodiment, said materials used to form both said fluid cavity and said foam cavity may share a common perimeter heat sealed seam.

In at least one exemplary embodiment, an exemplary system of the present disclosure is used in connection with a method, the method comprising the steps of opening the air valve and compressing the open cell or closed cell foam within the foam cavity, closing the air valve after compressing the open cell or closed cell foam, opening the fluid valve and introducing fluid into the fluid cavity, and closing the fluid valve after introducing fluid into the fluid cavity.

In at least one exemplary embodiment, the method further comprises the step of connecting tubing to the fluid valve and to a patient such that the fluid from the fluid cavity can pass through the fluid valve, into the tubing, and into the patient.

In at least one exemplary embodiment, the method further comprises the steps of opening the air valve to allow air to enter the foam cavity, allowing the open cell or closed cell foam to expand and exert pressure against the fluid cavity, and opening the fluid valve to allow the fluid to exit the fluid cavity due to the pressure exerted against the fluid cavity by the expanded open cell or closed cell foam.

In at least one exemplary embodiment of a pressurized sterile fluid storage bag system of the present disclosure, the system comprises a foam cavity formed from a rigid, non-conforming material and an elastic, conforming material and containing a resilient foam with capabilities to restore form when compressed, an air valve in communication with the foam cavity, a fluid cavity formed from said elastic, conforming material on one side and a material which may be rigid and non-conforming or elastic and conforming, and a fluid valve in communication with the fluid cavity, wherein when the resilient foam is compressed within the foam cavity and fluid is present within the fluid cavity, opening the air valve causes air to enter the foam cavity causing the resilient foam to expand and exert pressure against the elastic, conforming material, causing the fluid to exit the fluid cavity when the fluid valve is opened.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows an isolated perspective view of portions of a system in a pressurized configuration, according to an exemplary embodiment of the present disclosure;

FIG. 2 shows a cross-sectional exterior view of a system when it is in its pressurized configuration. according to an exemplary embodiment of the present disclosure;

FIG. 3 shows a cross-sectional view of a system when it is in its pressurized configuration with the interior foam compressed, the fluid cavity filled, and both the air inlet valve and the fluid outlet valve closed, according to an exemplary embodiment of the present disclosure;

FIG. 4 shows a cross-sectional view of a system when it is in its non-pressurized configuration with the interior foam uncompressed, the fluid cavity unfilled, and both the air inlet valve and the fluid outlet valve open. according to an exemplary embodiment of the present disclosure;

FIG. 5 shows an isolated cross-sectional view of an air inlet valve in its closed configuration/orientation, according to an exemplary embodiment of the present disclosure; and

FIG. 6 shows an isolated cross-sectional view of the fluid inlet valve in its closed configuration/orientation, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

For the purpose of explanation, the terms “front”, “back”, “left”, “right”, “upper”, “lower”, “top”, “bottom”, and similar terms shall correspond to the device as positioned in the specified figures. The device may assume alternate orientations and sizings other than those shown. It is understood that the device characterized in the attached drawings and described thereafter are exemplary orientations of the innovative concepts defined in the claims section of this patent. Hence, the specific characteristics shown are not to be considered limiting unless explicitly stated.

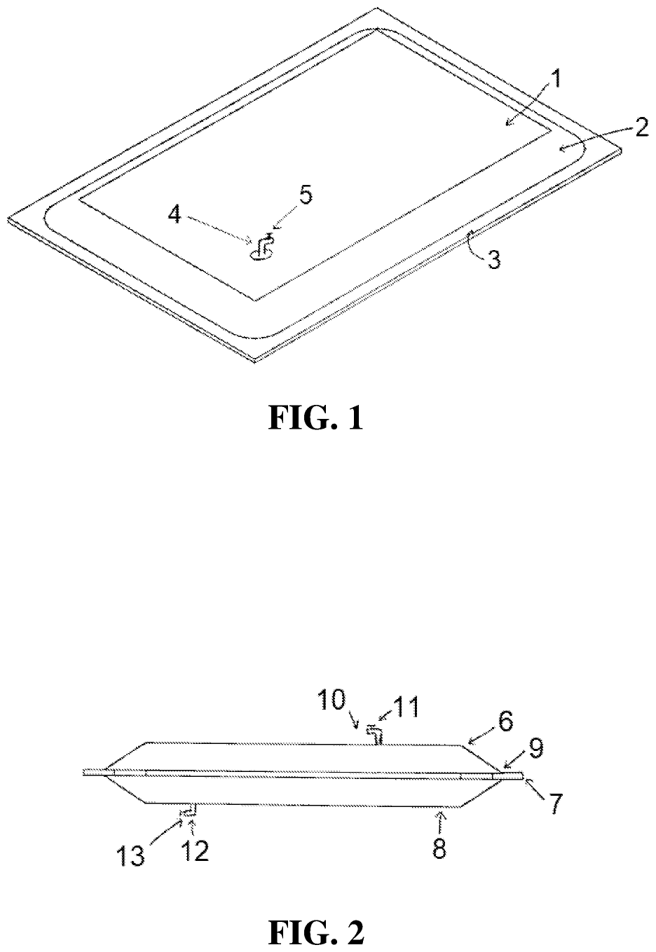

With reference to the figures, the present disclosure includes disclosure of embodiments of a storage bag system 100 and methods of using the same. As shown in FIG. 1, an isometric view of an exemplary storage bag of the present disclosure is shown comprising a foam insert 1, whereby foam insert 1 is made of (comprises) a rigid, non-conforming material, so that said material resists bending or otherwise changing from its native configuration under pressure. A foam insertion 2, as shown in FIG. 1, is positioned adjacent to, or relatively adjacent to, said foam insert 1, noting that not all embodiments of storage bags 100 of the present disclosure require a foam insert 1, as discussed in further detail. The size of the encapsulating foam insert 1 can be slightly larger or smaller than or the same size as than a corresponding foam insertion 2, configured such that it can be sealed on a relative perimeter 3 of storage bag system 100 in embodiments that utilize such a sealing element. In general, foam insert 1 must be sized and shaped, compressed or not, to fit within a space defined within storage bag system 100.

FIG. 1 also shows an air valve 4 and a corresponding air locking mechanism 5, whereby air valve 4 is in communication with an interior space (referred to herein as foam cavity 6, noted below) within storage bag system 100 so that, for example, air can be released from within storage bag system 100, and so that air can enter storage bag system 100 from its outside environment or another source of air. Air locking mechanism 5 is therefore configured to control the movement of air in and out of storage bag system 100, such that if space (foam cavity 6) within storage bag system 100 is under less pressure than its environment, opening air locking mechanism 5 would allow air from the outside to enter the inside of storage bag system 100, and should the space (foam cavity 6) within storage bag system 100 be at a higher pressure than its environment, opening air locking mechanism 5 would allow air from inside storage bag system 100 to escape.

FIG. 2 shows a cross-sectional side view of an exemplary storage bag system 100 in its pressurized configuration. As shown therein, a foam cavity 6, configured to encapsulate foam insertion 2, is directly adjacent to fluid cavity 8, with the dividing barrier 7 separating foam cavity 6 from fluid cavity 8. Dividing barrier 7, which is to be made of an elastic or otherwise conforming material, is positioned between foam cavity 6 and fluid cavity 8. Storage bag system 100 materials used to form an overall shape may share a common seam 9, such as shown in FIG. 2.

An air valve 10 (also referred to herein as air valve 4) is shown with its air locking mechanism 11 (also referred to herein as air locking mechanism 5) in its closed position to prevent air inflow to the foam cavity 6. FIG. 2 also shows a fluid valve 12 with its fluid locking mechanism 13 in its closed position to prevent fluid flow out of the fluid cavity 8. Fluid valve 12 is in communication with an interior space (referred to herein as fluid cavity 8) within storage bag system 100 so that, for example, fluid can be released from within storage bag system 100, and so that fluid can enter storage bag system 100 as desired. Fluid locking mechanism 13 is therefore configured to control the movement of fluid in and out of storage bag system 100, such that if fluid is to be transferred into fluid cavity 8, fluid locking mechanism 13 can be opened to allow said transfer and closed when said transfer is complete. Should it be desired to allow fluid from within fluid cavity 8 to flow out of fluid cavity 8, fluid locking mechanism 13 can be opened to allow said fluid flow to occur.

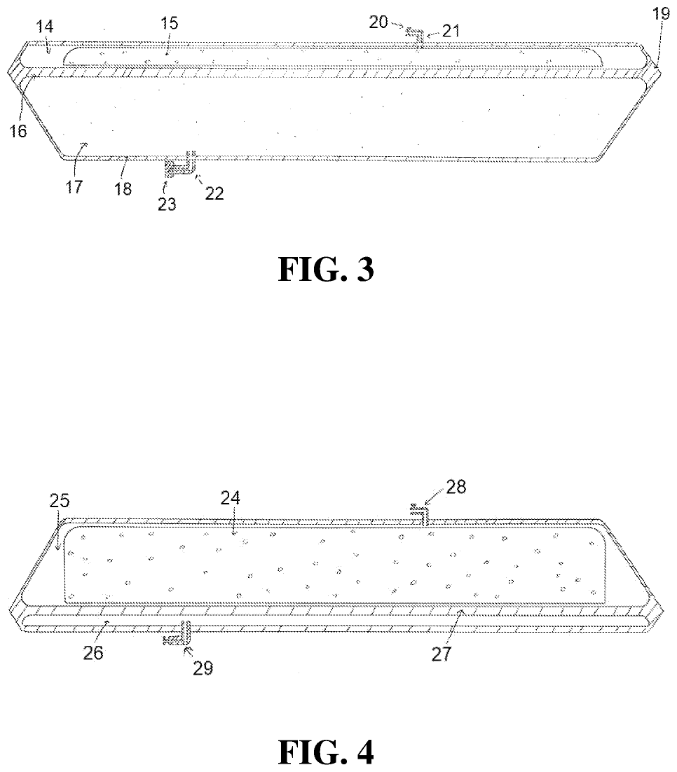

FIG. 3 shows a cross-sectional view of an exemplary storage bag system 100 in its pressurized configuration, whereby air from inside foam cavity 14 (also referred to herein as foam cavity 6) has been removed, such as by way of compression of storage bag system 100 and/or via some sort of vacuum so to compress foam insert 15 within said foam cavity 14. Foam cavity 14 is shown with minimal airspace and filled with foam insert 15. Foam insert 15 may be open-cell or closed-cell foam, and of variable porosity, so that said foam insert 15 can be compressed, such as shown in FIG. 3, and ultimately uncompressed, as shown in FIG. 4, the latter of which being or close to being a native state of foam insert 15.

Foam characteristics are dependent on force necessary to pressurize fluid within fluid cavity 17. Foam cavity 14 is separated from the fluid cavity 17 (also referred to herein as fluid cavity 8) by way of a dividing barrier 16 (also referred to herein as dividing barrier 7), which is to be made of an elastic, conforming material. Fluid cavity 17 is shown full of sterile liquid, and is closed from the ambient (outside environment) by the fluid barrier 18, which is to be made of a material that may be elastic and conforming or rigid and non-conforming. Fluid valve 22 (also referred to herein as fluid valve 12) allows for passage of fluid out of fluid cavity 17. FIG. 3 shows fluid locking mechanism 23 of fluid valve 22 in its locked position, with the exit/opening of fluid valve 22 fully covered/closed.

FIG. 3 also shows an air valve 21 (also referred to herein as air valves 4 and 10) in a closed configuration, by way of closing an air locking mechanism 21 (also referred to herein as air locking mechanisms 5 and 11), so that air cannot enter foam cavity 14. A common seam 19 is also shown around a relative perimeter of storage bag system 100.

FIG. 4 shows a cross-sectional view of an exemplary storage bag system 100 in its unpressurized configuration, whereby air is permitted to enter foam cavity 25 (also referred to herein as foam cavities 6 and 14) by way of air valve 28 (also referred to herein as air valves 10 and 21). As shown in FIG. 4, fluid valve 29 (also referred to herein as fluid valves 12 and 22) and air valve 28 (also referred to herein as air valves 4, 10, and 21) are in their open position, so that air is permitted to enter foam cavity 25 (also referred to herein as foam cavities 6 and 14), and so that fluid from within fluid cavity 26 (also referred to herein as fluid cavities 8 or 17) is permitted to exit the bag, such as to be introduced into a patient intravenously, for example. Foam insert 24 (also referred to herein as foam inserts 1 and 15) is shown in its uncompressed state in FIG. 4 due to air being allowed to enter foam cavity 25 and foam insert 24 so that foam insert 24 is permitted to expand, by way of air valve 28 being open. Foam cavity 25 is shown with residual air also due to air valve 28 being in its open configuration. Dividing barrier 27 (also referred to herein as dividing barriers 7 and 16) is shown compressing fluid cavity 26, such that there is no or little residual fluid content left in fluid cavity 26.

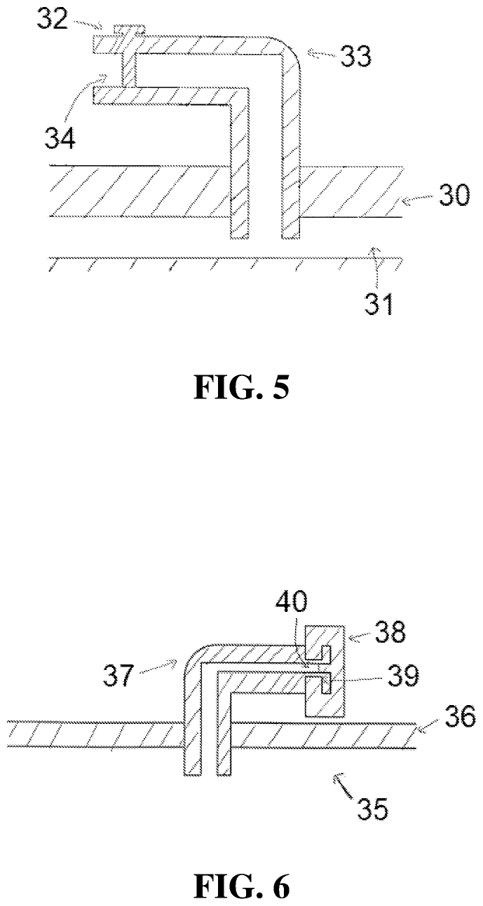

FIG. 5 shows a cross-sectional view of an air valve 33 (also referred to herein as air valves 4, 10, 21, and 28) and portions of a storage bag system 100, with air valve 33 shown in its closed configuration (such as by way of air locking mechanism 32, also referred to herein as air locking mechanisms 5, 11, and 20). Air valve 33 is shown as being in communication with foam cavity 31 (also referred to herein as foam cavities 6, 14, and 25), passing through fluid barrier 30 (also referred to herein as fluid barrier 18). When closed, air locking mechanism 32 prevents air to flow from the outside environment, through air valve opening 34, into air valve 33, and into foam cavity 31 to allow foam insert 1, 15, 24 to expand and apply pressure on dividing barrier 7, 16, 27, to cause fluid from fluid cavity 8, 17, 26 to exit fluid valve 12, 22, 29. Air valve 33, in various embodiments, can be configured as having a relatively low profile, such as shown in FIG. 5, such that air flow through portions of air valve 33 is relatively perpendicular to air flow from foam cavity 31 into air valve 33.

FIG. 6 shows a cross-sectional view of a fluid valve 37 (also referred to herein as fluid valves 12, 22, and 29) and portions of a storage bag system 100, with fluid valve 37 shown in its closed configuration (such as by way of fluid locking mechanism 38, also referred to herein as fluid locking mechanisms 13 and 23). Fluid valve 37 is shown as being in communication with fluid cavity 35 (also referred to herein as fluid cavities 8, 17, and 26), passing through fluid barrier 36 (also referred to herein as fluid barriers 18 and 30). When closed, fluid locking mechanism 38 prevents fluid to flow from inside fluid cavity 35, through fluid valve opening 40, into fluid valve 37, and out of fluid valve 37. Fluid valve 37, in various embodiments, can be configured as having a relatively low profile, such as shown in FIG. 6, such that fluid flow through portions of fluid valve 37 is relatively perpendicular to fluid flow from fluid cavity 35 into fluid valve 37. Locking mechanism 38 closes off the fluid valve 37 such that fluid may not flow through ambient opening 40 of fluid valve 37. A luer lock 39 on an outer edge of fluid valve 37 allows for tubing or other instruments to be fluidly connected to fluid valve 37.

While various embodiments of systems and devices and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

The foregoing disclosure of specific embodiments is intended to be illustrative of the broad concepts comprehended by the present disclosure.

Reference List

U.S. Pat. No. 2,608,320 to Harrison, Jr.

U.S. Pat. No. 3,838,794 to Cogley, et al.

U.S. Pat. No. 3,871,377 to Treace

U.S. Pat. No. 3,949,753 to Dockhorn

U.S. Pat. No. 4,098,434 to Uhlig, Albert R.

U.S. Pat. No. 4,539,004 to Eckenhoff, et al.

U.S. Pat. No. 5,163,909 to Stewart

U.S. Pat. No. 5,176,641 to Idriss

U.S. Pat. No. 5,205,820 to Kriesel

U.S. Pat. No. 5,497,912 to Hoback, et al.

U.S. Pat. No. 5,810,202 to Hoback, et al.

U.S. Pat. No. 8,992,489 to Spohn, et al.

U.S. Patent Application Publication No. 2003/0135159 of Daily, et al.

1. A pressurized sterile fluid storage bag system, the system comprising:
a foam cavity formed from a rigid, non-conforming material and an elastic, conforming material and containing a resilient open cell or closed cell foam with capabilities to restore form when compressed;
an air valve;
a fluid cavity formed from said elastic, conforming material on one side and a material which may be rigid and non-conforming or elastic and conforming; and
a fluid valve.

2. The system of claim 1, further comprising:
a common seam to seal the perimeter of all cavity forming materials, such that said fluid cavity is adjacent to said foam cavity with the elastic, conforming material separating the two.

3. The system of claim 1, wherein the said foam cavity is shaped such that the foam insert is not compressed when said air valve is open.

4. The system of claim 1, wherein said foam cavity can be compressed in a matter which compresses its foam contents.

5. The system of claim 1, wherein said rigid, nonconforming material forming said foam cavity does not stretch during expansion of foam insert contents.

6. The system of claim 1, wherein said air valve passes through the rigid, nonconforming material used to create said foam cavity and allows air passage between the ambient and the internal air space of said foam cavity.

7. The system of claim 1, wherein said air valve features a locking mechanism which allows for complete air locking to fully disallow or permit passage of air between ambient and internal air space of said foam cavity.

8. The system of claim 1, wherein said air valve features a locking mechanism which allows the user to adjust the diameter of air exit hole.

9. The system of claim 1, wherein said elastic, nonconforming material acts as the dividing barrier between said foam cavity and said fluid cavity.

10. The system of claim 1, wherein said elastic, nonconforming material is able to stretch during expansion of said foam insert.

11. The system of claim 1, wherein said fluid cavity is air tight and compresses in internal volume during expansion of said foam insert.

12. The system of claim 1, wherein said fluid valve passes through the rigid, nonconforming material or elastic, conforming material used to form the outer surface of said fluid cavity and allows for fluid passage between the ambient and internal space of said fluid cavity.

13. The system of claim 1, wherein said fluid valve features a luer lock on its exterior portion to allow for connection to tubing or various instruments.

14. The system of claim 1, wherein said fluid valve features a locking mechanism which allows for complete air locking to fully disallow or permit passage of fluid between the ambient and internal space of said fluid cavity.

15. The system of claim 1, wherein said fluid valve features a locking mechanism which allows the user to adjust the diameter of the fluid exit hole.

16. The system of claim 1, wherein said materials used to form both said fluid cavity and said foam cavity may share a common perimeter heat sealed seam.

17. The system of claim 1, used in connection with a method, the method comprising the steps of:
opening the air valve and compressing the open cell or closed cell foam within the foam cavity;
closing the air valve after compressing the open cell or closed cell foam;
opening the fluid valve and introducing fluid into the fluid cavity; and
closing the fluid valve after introducing fluid into the fluid cavity.

18. The method of claim 17, further comprising the step of:
connecting tubing to the fluid valve and to a patient such that the fluid from the fluid cavity can pass through the fluid valve, into the tubing, and into the patient.

19. The method of claim 18, further comprising the steps of:
opening the air valve to allow air to enter the foam cavity, allowing the open cell or closed cell foam to expand and exert pressure against the fluid cavity; and
opening the fluid valve to allow the fluid to exit the fluid cavity due to the pressure exerted against the fluid cavity by the expanded open cell or closed cell foam.

20. A pressurized sterile fluid storage bag system, the system comprising:
a foam cavity formed from a rigid, non-conforming material and an elastic, conforming material and containing a resilient foam with capabilities to restore form when compressed;
an air valve in communication with the foam cavity;
a fluid cavity formed from said elastic, conforming material on one side and a material which may be rigid and non-conforming or elastic and conforming; and
a fluid valve in communication with the fluid cavity;
wherein when the resilient foam is compressed within the foam cavity and fluid is present within the fluid cavity, opening the air valve causes air to enter the foam cavity causing the resilient foam to expand and exert pressure against the elastic, conforming material, causing the fluid to exit the fluid cavity when the fluid valve is opened.
